(19) United States
(12) Patent Application Publication
(10) Pub. No.: US 2018/0078256 A1
(43) Pub. Date: Mar. 22, 2018

(54) MEDICAL STAPLER

(71) Applicant: OLYMPUS CORPORATION, Tokyo (JP)

(72) Inventors: Kayuri KIMURA, Saitama (JP); Masayuki KOBAYASHI, Tokyo (JP); Yoshiyuki KUMADA, Tokyo (JP); Hiroshi OKABE, Tokyo (JP); Keisuke TSURIMOTO, Hyogo (JP)

(73) Assignee: OLYMPUS CORPORATION, Tokyo (JP)

(21) Appl. No.: 15/823,872

(22) Filed: Nov. 28, 2017

Related U.S. Application Data
(63) Continuation of application No. PCT/JP2015/066794, filed on Jun. 10, 2015.

(51) Int. Cl. A61B 17/072

(52) U.S. Cl. CPC A61B 17/07207; A61B 2090/034; A61B 2017/07228; A61B 2017/07285

(57) ABSTRACT

A medical stapler including: a first jaw that has a plurality of staples arrayed in a row at intervals; a second jaw that has a second gripping surface that sandwiches living tissue between the second gripping surface and a first gripping surface; and a knife that moves between the first gripping surface and the second gripping surface along the row of staples from the one end of the row to a position extended from the other end of the row of staples, thus cutting the living tissue.

### CROSS-REFERENCE TO RELATED APPLICATIONS

This is a continuation of International Application PCT/JP2015/066794, with an international filing date of Jun. 10, 2015, which is hereby incorporated by reference herein in its entirety.

### TECHNICAL FIELD

The present invention relates to a medical stapler.

### BACKGROUND ART

In surgical operations, medical staplers capable of suturing and cutting living tissue have been conventionally used (for example, see PTLs 1, 2, and 3). With such medical staplers, living tissue can be cut, with a knife, along one side of a suture line of staples.

### CITATION LIST

### Patent Literature

{PTL 1} Publication of Japanese Patent No. 4257270

{PTL 2} Publication of Japanese Patent No. 3926831

{PTL 3} Japanese Unexamined Patent Application, Publication No. 2012-110674

### SUMMARY OF INVENTION

An object of the present invention an object thereof is to provide a medical stapler capable of cutting living tissue in one step.

### Solution to Problem

According to one aspect, the present invention provides a medical stapler including: a first jaw that has a first gripping surface and that has a plurality of staples arrayed in a row at intervals; a second jaw that is opposed to the first gripping surface and that has a second gripping surface that sandwiches living tissue between the second gripping surface and the first gripping surface; and a knife that moves between the first gripping surface and the second gripping surface along the row of staples from one end of the row of staples to a position extended from the other end of the row of staples, thus cutting the living tissue.

### BRIEF DESCRIPTION OF DRAWINGS

FIG. 1A is a side view showing the configuration of a suture part of a medical stapler according to one embodiment of the present invention.

FIG. 1B is a side view showing the suture part shown in FIG. 1A in a state in which jaws are closed.

FIG. 2A is a plan view of a first jaw of the suture part shown in FIG. 1A viewed from a first gripping surface side.

FIG. 2B is a sectional view of the suture part shown in FIG. 2A cut along the line IIa-IIa.

FIG. 2C is a sectional view of the suture part shown in FIG. 2A cut along the line IIb-IIb.

FIG. 3 is a view showing living tissue sutured and cut by the suture part shown in FIG. 1A.

FIG. 4A is a plan view showing a modification of the first jaw.

FIG. 4B is a side view showing a suture part provided with the first jaw shown in FIG. 4A.

FIG. 5A is a plan view showing another modification of the first jaw.

FIG. 5B is a side view showing a suture part provided with the first jaw shown in FIG. 5A.

FIG. 6A is a plan view showing still another modification of the first jaw.

FIG. 6B is a sectional view of a suture part provided with the first jaw shown in FIG. 6A cut along the line VI-VI and is a view showing a state in which non-slip parts are accommodated in slots.

FIG. 6C is a sectional view of the suture part provided with the first jaw shown in FIG. 6A cut along the line VI-VI and is a view showing a state in which the non-slip parts are pushed out from the slots.

FIG. 7 is a perspective view showing still another modification of the suture part shown in FIG. 1A.

### DESCRIPTION OF EMBODIMENT

A medical stapler 1 according to one embodiment of the present invention will be described below with reference to the drawings.

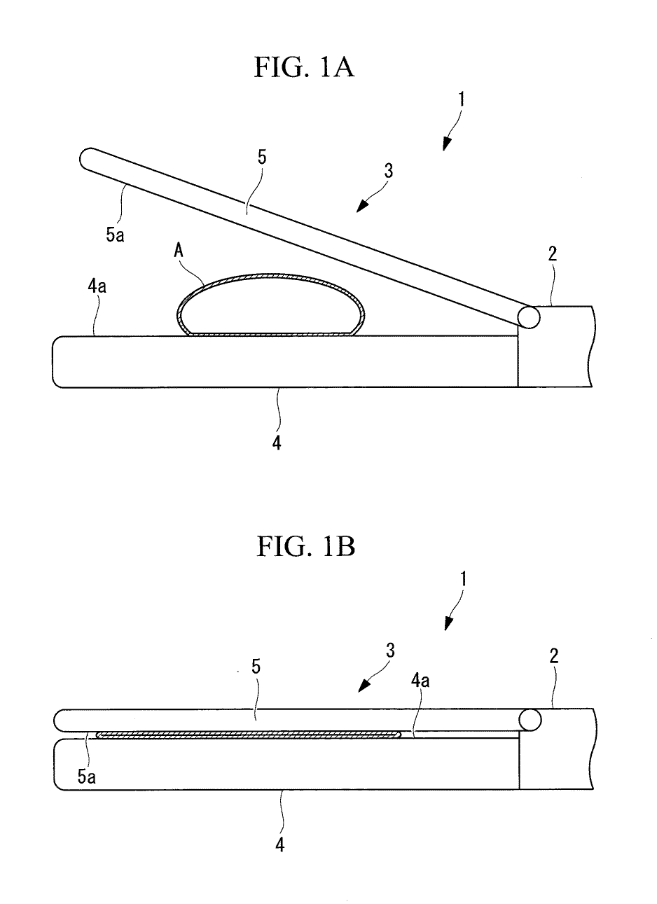

As shown in FIGS. 1A and 1B, the medical stapler 1 of this embodiment is provided with: an elongated insertion portion 2 that can be inserted into a body; a suture part 3 that is provided at a distal end of the insertion portion 2; and an operating part (not shown) that is connected to a base end of the insertion portion 2.

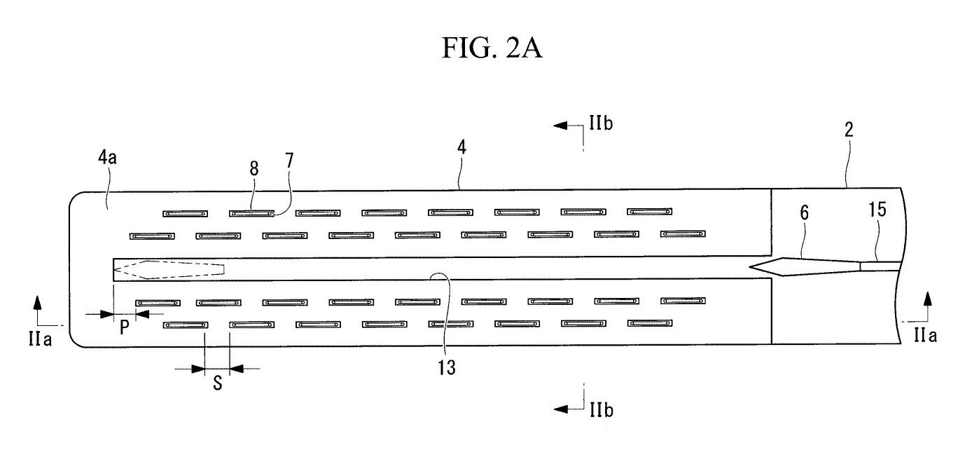

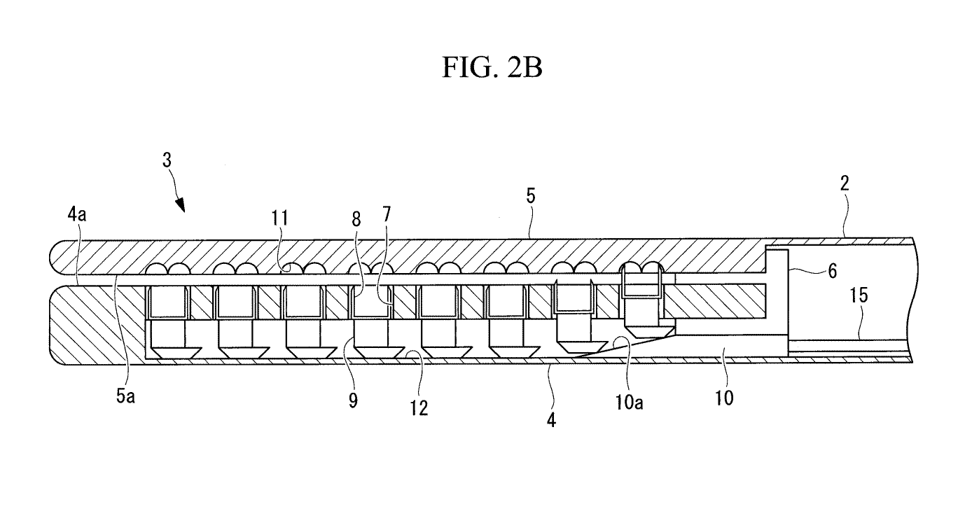

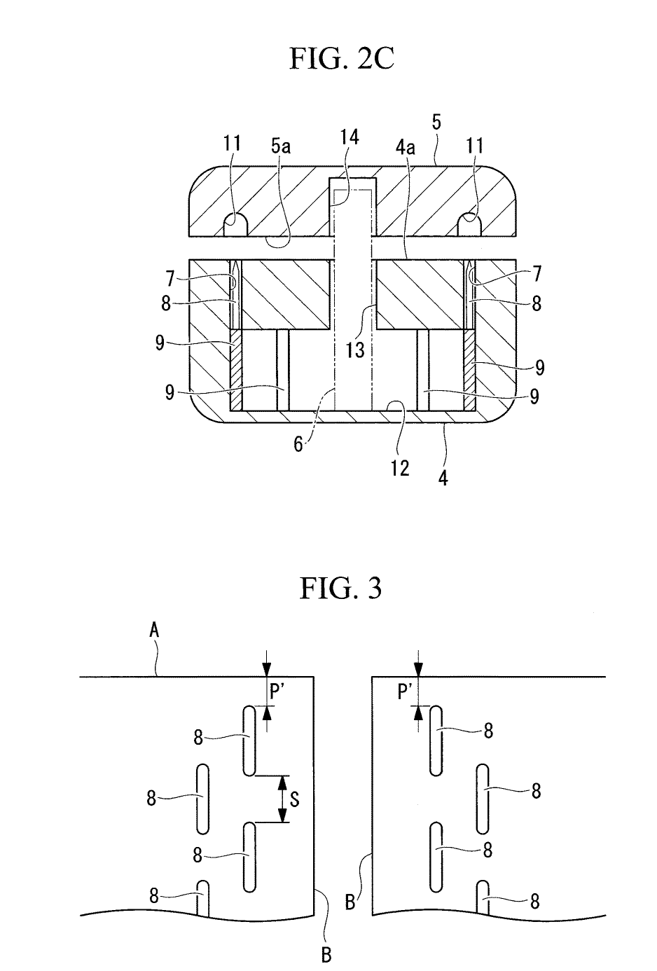

The suture part 3 is provided with: a first jaw 4 and a second jaw 5 that are coupled in an openable/closable manner; and a knife 6 (see FIGS. 2A to 2C) that cuts living tissue A sandwiched between the first jaw 4 and the second jaw 5. The first jaw 4 and the second jaw 5 have each a substantially rectangular shape extending in the longitudinal direction of the insertion portion 2 and are coupled in a manner allowing base end portions thereof to swivel relative to each other so as to open and close distal ends thereof. The first jaw 4 and the second jaw 5 are driven to open/close when an operator operates an open/close operation member (not shown), such as a rotating knob or a lever, provided on the operating part. The first jaw 4 and the second jaw 5 have, respectively, a first gripping surface 4a and a second gripping surface 5a that are opposed to each other with a gap for sandwiching the living tissue A therebetween.

As shown in FIGS. 2A to 2C, the first jaw 4 holds, therein, staples 8 that are accommodated in staple slots 7 formed in the first gripping surface 4a, pushers 9 that push the staples 8 from the insides of the staple slots 7, and a cam member 10 that drives the pushers 9. In FIG. 2A, the cam member 10 is not shown.

As shown in FIG. 2A, the staple slots 7 are arrayed in a row at intervals in the longitudinal direction of the first gripping surface 4a. The staple slots 7 are formed in two rows or more (in this example, four rows) parallel to each other. The staples 8 are formed of thin lines bent in a U-shaped manner and are supported in the staple slots 7 in a manner allowing them to be injected from openings of the staple slots 7 with two ends thereof being directed toward the second jaw 5. As shown in FIG. 2B, anvil pockets 11 that are dents for deforming the staples 8 into a substantially B-shape are formed on the second gripping surface 5a at positions opposing the staple slots 7.

A path 12 that extends parallel to the rows of the staple slots 7 and that communicates with the staple slots 7 is formed on the opposite sides of the staple slots 7 from the first gripping surface 4a. The pushers 9 are disposed in the path 12 in contact with the staples 8.

The cam member 10 is disposed at the base end of the rows of the pushers 9 and is provided so as to be movable in the path 12 in the longitudinal direction. A distal-end surface of the cam member 10 is an inclined surface 10a that is inclined with respect to the first gripping surface 4a. While the cam member 10 is moving in the path 12 from the base end toward the distal end, the inclined surface 10a sequentially pushes up the pushers 9 starting from those disposed close to the base end, toward the staples 8 side, thereby pushing out the staples 8 from the insides of the staple slots 7. Accordingly, the staples 8 are sequentially injected starting from those disposed close to the base end.

The cam member 10 is driven when the operator operates an extrusion operation member (not shown), such as a rotating knob or a lever, provided in the operating part. Reference sign 15 denotes a shaft-like or cable-like drive member that passes inside the insertion portion 2 and that couples the cam member 10 and the operating part. When the drive member 15 is moved in the longitudinal direction according to an operation of the extrusion operation member performed by the operator, the cam member 10 is moved in the path 12.

As shown in FIG. 2C, a knife slot 13 that extends, between two rows of the staple slots 7, from the base end of the first gripping surface 4a parallel to the rows of the staple slots 7 and that guides the knife 6 is formed in the first gripping surface 4a. The knife 6 is provided so as to be movable in the knife slot 13 in a state in which a knife point thereof is directed to the distal end, and a part thereof is protruded from the first gripping surface 4a. A knife slot 14 is formed on the second gripping surface 5a at a position opposing the knife slot 13, and, in a state in which the jaws 4 and 5 are closed, an edge of the knife 6 on the second jaw 5 side is inserted into the knife slot 14. The cam member 10 is brought into contact with or is fixed to an edge of the knife 6 on the first jaw 4 side, so that the knife 6 is moved together with the cam member 10.

The knife slot (movable-range defining means) 13, which is formed in the first gripping surface 4a, is terminated at a position (limit position) extended further toward the distal end than the distal end of the four rows of the staple slots 7 by a predetermined distance P. Therefore, the movable range of the knife 6 is limited by the terminal position of the knife slot 13, and the knife 6 can be moved further toward the distal end than the distal end of the rows of the staple slots 7. The distance P in the longitudinal direction between the terminal of the knife slot 13 and the distal end of the staple 8 that is located closest to the distal end is equal to or less than the width S of a gap between two staples 8 accommodated in the two staple slots 7 adjacent in the longitudinal direction.

Next, the operation of the thus-configured medical stapler 1 will be described by taking an example case in which the tubular living tissue A is sutured and cut.

In order to suture and cut the living tissue A by using the medical stapler 1 of this embodiment, the living tissue A is sandwiched between the pair of jaws 4 and 5 so as to intersect the jaws 4 and 5 (see FIG. 1A), and the pair of jaws 4 and 5 are closed, thereby gripping the living tissue A between the gripping surfaces 4a and 5a in a state in which the living tissue A is flattened in the radial direction (see FIG. 1B). At this time, in order to prevent the living tissue A from being made to protrude further toward the distal end than the knife slot 13, the living tissue A is positioned with respect to the gripping surfaces 4a and 5a.

Next, the cam member 10 is moved from the base end toward the distal end. Accordingly, the staples 8, starting from those disposed close to the base end, are sequentially injected from the insides of the staple slots 7 toward the living tissue A. The two ends of each injected staple 8 that have passed through the living tissue A are brought into contact with the anvil pocket 11 and are bent, thereby being deformed into a substantially B-shape. Accordingly, four rows of suture lines that are formed of rows of the staples 8 are formed on the living tissue A.

When the knife 6 is moved from the base end toward the distal end in association with the cam member 10, the living tissue A is cut between the two suture lines. Accordingly, the living tissue A is cut while being sutured, and, as shown in FIG. 3, two cut ends B of the living tissue A are sutured with the staples 8 so as to be occluded.

Since the knife 6 can move further toward the distal end than the distal end of the rows of the staples 8, the living tissue A can be cut, with the knife 6, up to a position beyond the terminals of the suture lines. Accordingly, there is an advantage in that the living tissue A can be cut over the full width in one step without leaving part of the living tissue A located near terminal portions of the suture lines uncut. The protruding amount P of the knife 6 from the terminals of the suture lines is equal to or less than the width S of a gap between the staples 8. Therefore, a distance P′ from the terminals of the suture lines to the terminal of a cutting line of the knife 6 is also equal to or less than the width S. Accordingly, there is an advantage in that equivalent joining forces to the suture forces in the suture lines are ensured at end portions of the cut ends B, which are located further toward the distal end than the terminals of the suture lines are, thereby making it possible to tightly occlude the living tissue A so as to prevent leakage of the content of the living tissue A.

During this stapling work, if the wall of a lumen is sutured and cut off from the inside of the lumen, operation of an endoscope for performing observation is also required, thus increasing not only the complexity but also the treatment difficulty and the treatment time, and the impact thereof is large. According to this embodiment, with the above-described configuration, such an impact on the treatment difficulty and the treatment time can be suppressed.

In this embodiment, although the movable range of the knife 6 is defined by the terminal position at the distal end of the knife slot 13, instead of this, the movable range of the knife 6 may be defined by limiting the movable range of the drive member 15 in the longitudinal direction.

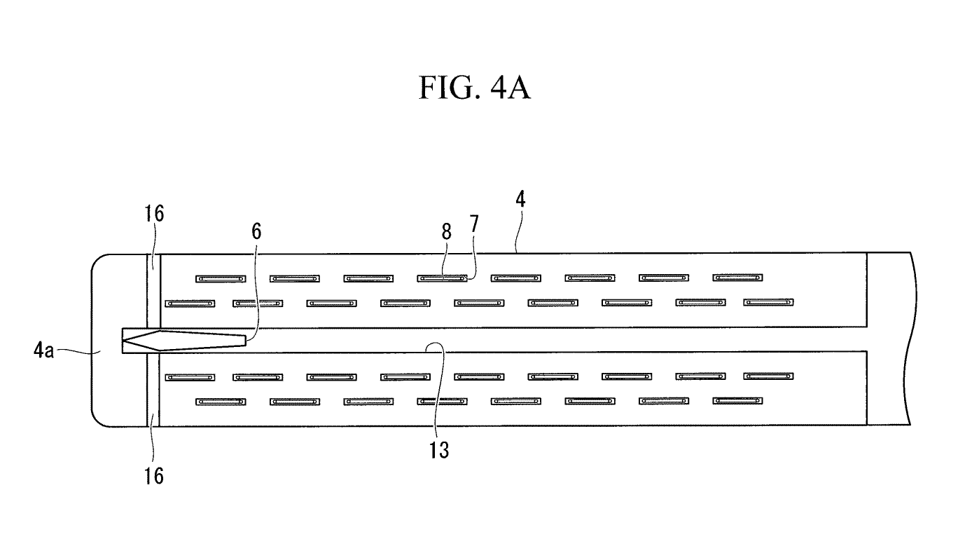

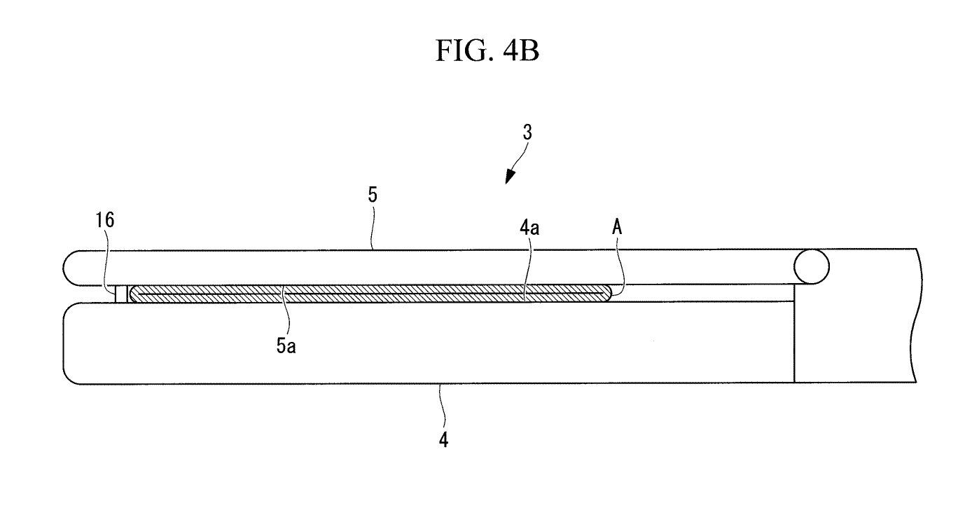

In this embodiment, as shown in FIGS. 4A and 4B, living-tissue locking parts 16 that prevent the movement of the living tissue A in the longitudinal direction with respect to the first gripping surface 4a may be provided on the first gripping surface 4a. In FIG. 4A, the cam member 10 and the drive member 15 are not shown.

The living-tissue locking parts 16 are members projecting from the first gripping surface 4a and have a height reaching the second gripping surface 5a in a state in which the pair of jaws 4 and 5 are closed. The living-tissue locking parts 16 are located further toward the distal end than the distal ends of the staple slots 7 that are located closest to the distal end are and are preferably provided over the full width of the first gripping surface 4a in a direction intersecting the longitudinal direction.

The width of a gap between the distal ends of the staple slots 7 that are located closest to the distal end and the living-tissue locking parts 16 is equal to or less than the width S of a gap between the staples 8 in the two staple slots 7 adjacent in the longitudinal direction. Accordingly, part of the living tissue A disposed further toward the base end than the living-tissue locking parts 16 cannot be moved further toward the distal end than the living-tissue locking parts 16, is held within the range in which it can be sutured with the staples 8, and is thus reliably sutured. In this modification, the knife slot 13 may extend further toward the distal end than the living-tissue locking parts 16 are.

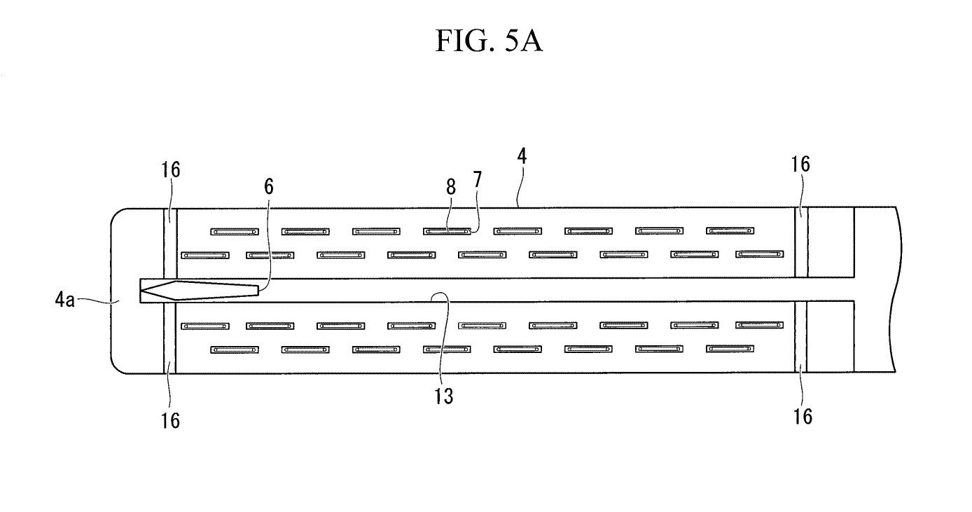

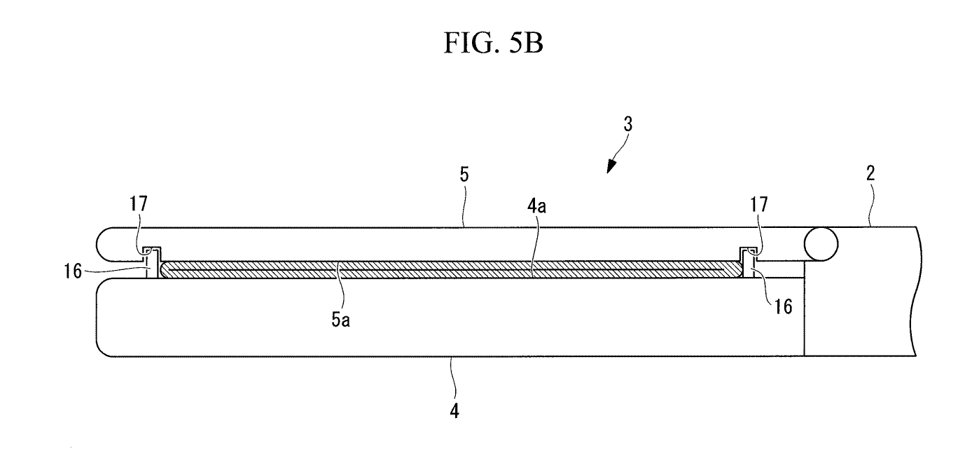

As shown in FIGS. 5A and 5B, the living-tissue locking parts 16 may also be provided closer to the base end than the rows of the staple slots 7 are. Holes 17 into which end portions of the living-tissue locking parts 16 are fitted may be formed on the second gripping surface 5a at positions opposing the living-tissue locking parts 16. The living-tissue locking parts 16 may be provided on the second gripping surface 5a instead of the first gripping surface 4a. In FIG. 5A, the cam member 10 and the drive member 15 are not shown.

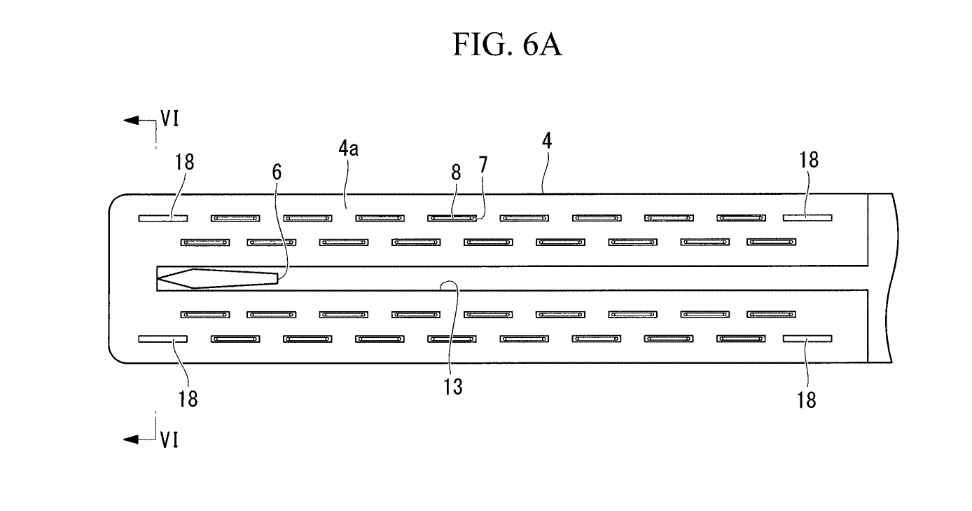

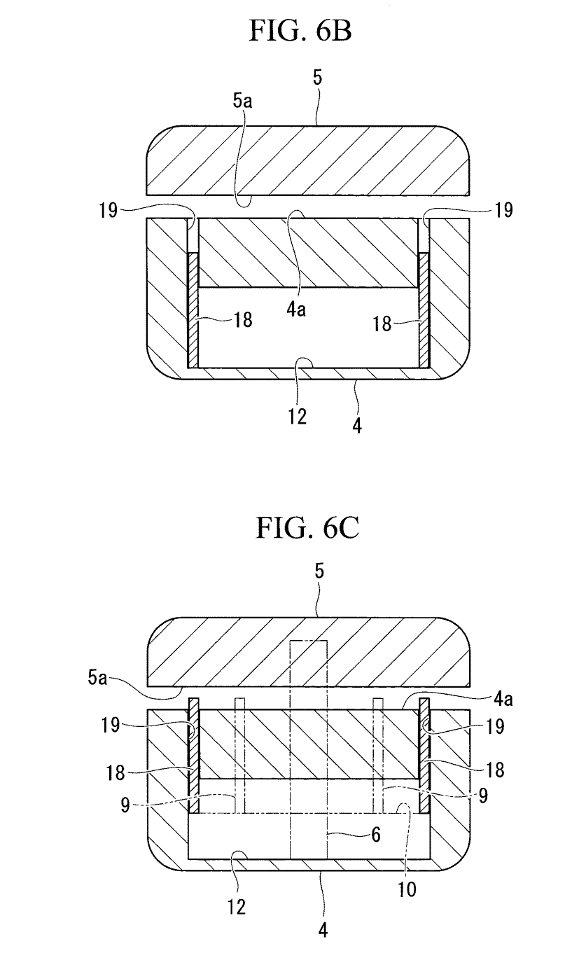

In this embodiment, as shown in FIG. 6A, non-slip parts 18 for holding the living tissue A may be provided at least at positions closer to the distal end than the rows of the staple slots 7 are, preferably, at positions closer to the distal end and the base end than they are. In FIG. 6A, the cam member 10 and the drive member 15 are not shown.

The end portions of the pushers 9 that have pushed out the staples 8 project from the staple slots 7 and press the living tissue A toward the second gripping surface 5a. Accordingly, the living tissue A is pressed against the second gripping surface 5a, thus preventing the living tissue A from slipping with respect to the second gripping surface 5a. The non-slip parts 18 are members projecting from the first gripping surface 4a by substantially the same amount as the pushers 9, so as to give the living tissue A the pressing force equivalent to those of the individual pushers 9. Accordingly, at the distal end of the rows of the staple slots 7, the living tissue A is prevented from slipping and can be more reliably cut.

Although the non-slip parts 18 may be fixed in the first gripping surface 4a, as shown in FIG. 6B, the non-slip parts 18 may be accommodated in slots 19 formed in the first gripping surface 4a in a manner allowing them to project from the slots 19, like the staples 8. As shown in FIG. 6C, the non-slip parts 18 in the slots 19 are pushed out from the insides of the slots 19 by the cam member 10.

By doing so, because the non-slip parts 18 are accommodated inside the first jaw 4 before the living tissue A is sutured and cut, the non-slip parts 18 can be prevented from interfering with the gripping operation of the living tissue A.

In this embodiment, although the straight-shaped jaws 4 and 5 are provided, instead of this, jaws that are curved in an arc-shaped manner or annular jaws may be provided.

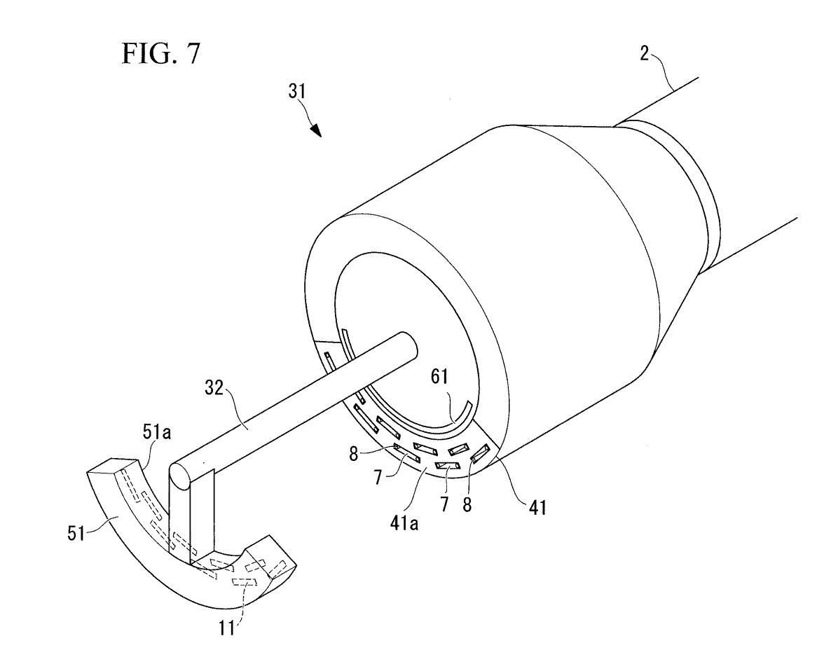

FIG. 7 shows an example suture part 31 that is provided with a pair of arc-shaped jaws 41 and 51. Reference sign 32 denotes a member for moving the second jaw 51 in directions close to and away from the first jaw 41. In an arc-shaped first gripping surface 41a, the staple slots 7 are formed in an array in the circumferential direction, and the staples 8 are simultaneously pushed out from the insides of the staple slots 7.

The knife 61 has a substantially semi-cylinder shape disposed concentrically with the rows of the staple slots 7, is supported by the first jaw 41 with the cutting edge thereof being directed toward the second jaw 51, and projects from the first gripping surface 41a substantially at the same time as when the staples 8 are pushed out. The knife 61 extends further toward both sides than both ends of the rows of the staple slots 7 in the circumferential direction, and both ends of the knife 61 in the circumferential direction are located at positions extended from both ends of the rows of the staple slots 7. Accordingly, the living tissue A can be cut, with the knife 61, up to positions extended from both ends of the suture line in the circumferential direction.

### REFERENCE SIGNS LIST

1 medical stapler
4, 41 first jaw
4a, 41a first gripping surface
5, 51 second jaw
5a, 51a second gripping surface
6, 61 knife
13 knife slot (movable-range defining means)
16 living-tissue locking part
18 non-slip parts

1. A medical stapler comprising:
a first jaw that has a first gripping surface and that has a plurality of staples arrayed in a row at intervals;
a second jaw that is opposed to the first gripping surface and that has a second gripping surface that sandwiches living tissue between the second gripping surface and the first gripping surface; and
a knife that moves between the first gripping surface and the second gripping surface along the row of staples from one end of the row of staples to a position extended from the other end of the row of staples, thus cutting the living tissue.

2. A medical stapler according to claim 1, further comprising a movable-range defining means that defines a movable range of the knife such that the knife does not move beyond a limit position extended from the other end of the row of staples by a predetermined distance in the longitudinal direction of the row of staples,
wherein the predetermined distance is equal to or less than the width of each of the intervals between two adjacent staples.

3. A medical stapler according to claim 2,
wherein at least one of the first gripping surface and the second gripping surface has a knife slot that extends along one side of the row of staples and that guides movement of the knife; and
the movable-range defining means is formed of the knife slot, which terminates at the limit position.

4. A medical stapler according to claim 1,
wherein one of the first gripping surface and the second gripping surface is provided with a living-tissue locking part that projects from the corresponding one of the first gripping surface and the second gripping surface to the other and that locks movement of the living tissue sandwiched between the first gripping surface and the second gripping surface, in a direction along the row of staples; and
the living-tissue locking part is provided at a position extended from the other end of the row of staples by a distance equal to or less than the width of each of the intervals between two adjacent staples, in the longitudinal direction of the row of staples.

5. A medical stapler according to claim 4,
wherein the first gripping surface or the second gripping surface has a non-slip part that projects from the corresponding first gripping surface or second gripping surface and that presses the living tissue sandwiched between the first gripping surface and the second gripping surface; and
the non-slip part is provided further outward than the other end of the row of staples is, while forming a row with the staples.

6. A medical stapler according to claim 1, wherein the non-slip part is provided in a projectable manner from the first gripping surface or the second gripping surface in association with the movement of the knife.

7. A medical stapler according to claim 1, wherein the knife extends along the row of staples, both ends of the knife are disposed at positions extended in directions along the row of staples from both ends of the row of staples, and the knife is provided in a projectable manner from one of the first gripping surface and the second gripping surface toward the other.
